(19) United States
(12) Patent Application Publication
(10) Pub. No.: US 2023/0310308 A1
(43) Pub. Date: Oct. 5, 2023

(54) Drug Delivery Systems, Apparatuses, and Methods

(71) Applicant: Marc Giroux, Lynnwood, WA (US)

(72) Inventor: Marc Giroux, Lynnwood, WA (US)

(21) Appl. No.: 18/123,251

(22) Filed: Mar. 17, 2023

Related U.S. Application Data
(60) Provisional application No. 63/408,338, filed on Sep. 20, 2022.
(60) Provisional application No. 63/320,701, filed on Mar. 17, 2022.

(51) Int. Cl. A61M 15/08; A61N 2/06; A61K 9/00

(52) U.S. Cl. CPC A61K 9/0009; A61K 9/0043; A61M 15/08; A61N 2/06; A61M 2209/088; A61M 2210/0693

(57) ABSTRACT

A system includes a nose-to-brain medication delivery device including a medication dispersion device and a magnetic field generator. The generator, when in use, generates a magnetic field of sufficient strength and direction to reposition an ionized and/or magnetized medication to a selected drug delivery site on the brain following initial delivery of the medication from the medication delivery device to the brain cavity via the nose while bypassing the blood-brain barrier.

### CROSS REFERENCE TO RELATED APPLICATIONS

This application claims the benefit under 35 U.S.C. § 119 of U.S. Provisional Pat. App. No. 63/320,701, filed on Mar. 17, 2022 and entitled “Drug Delivery Device and Method”, and to U.S. Provisional Pat. App. No. 63/408,338, filed on Sep. 20, 2022 and entitled “Drug Delivery Apparatus and Method”, each of which is incorporated herein by reference.

### BACKGROUND

Drugs are used for treatment of a large variety of conditions and diseases. Bioactive substances as drug candidates are regularly being discovered. Often, the ability to use a new drug requires delivery to a certain part of the body, but no effective delivery approach is known. Identifying new delivery approaches would be beneficial.

### BRIEF DESCRIPTION OF THE DRAWINGS

Some embodiments are described below with reference to the following accompanying drawings.

FIG. 1 is a cross-sectional view of a nasal cavity.

FIG. 2 is a top, magnified view of a neuron.

FIG. 3 is a cut-away, perspective view of one example of a carrier.

FIG. 4 shows a perspective view of one example of a medication delivery device.

FIG. 5 shows a perspective view of an example headgear.

FIGS. 6A-6B show a perspective view of another example headgear.

FIG. 7 shows a perspective view of the FIG. 5 head gear in an example combined with a grid.

FIGS. 8-10 show hypothetical imaging of repositioned medication on the brain using one magnet.

FIGS. 9-12 graphically depict repositioning medication on the brain using two magnets.

FIG. 13 is a perspective, magnified view of a multiple-walled nanotube.

### DETAILED DESCRIPTION

The example implementations described herein refer to the accompanying drawings. The same reference numerals in the various drawings may identify the same or similar features.

In some instances, a desire exists to target a site in the brain for delivery of a drug. Drugs are very often administered orally, followed by eventual uptake into the circulatory system for delivery to a selected site. Drugs are also administered intravenously for delivery to a selected site, also by the circulatory system. However, many drugs cannot be delivered in therapeutically-effective amounts across the blood-brain barrier. As a result, targeted delivery to the brain via the circulatory system is not feasible for some drugs.

Some examples described herein include repositioning the drug to the selected drug delivery site in the brain following nose-to-brain delivery while bypassing the blood-brain barrier. In some implementations, a medication dispersion device disperses a medication in a gas so that the medication may distribute in the nasal cavity for delivery to the brain cavity. The gas may be ambient air. Without being limited to any particular theory for transport of the medication to and in the brain cavity, it is believed that the medication enters the cerebral spinal fluid (CSF) via neuronal pathway at the olfactory region and the trigeminal nerve. If the medication is ionized and/or magnetized, then it may respond to a magnetic field. Accordingly, a magnetic field applied to the brain may reposition the medication to a selected drug delivery site on the brain.

Therefore, some implementations described herein provide a system, apparatus, and method for targeting a site in the brain for delivery of a drug. Drugs that cannot pass through the blood-brain barrier may nonetheless be targeted to the brain through intranasal administration. Specifically, selected parts of the brain where the drug may provide the most benefit may be targeted. Also, drugs that feasibly could pass through the blood-brain barrier and affect wide regions of the brain through the circulatory system, may instead be targeted through intranasal administration to selected parts of the brain where the drug may provide the most benefit.

A nose-to-brain drug delivery device can be designed to do drug delivery targeting in the brain using ionized, cationic, and/or magnetized medications in accompaniment with an external magnetic field to attract and/or drive the medication to the desired target drug delivery site. As the term is used herein, “medication” refers to a drug or a combination of a drug and a carrier that is linked to, encapsulates, otherwise carries the drug, or a combination thereof. For example, a carrier may be linked to a drug by a covalent bond, an ionic bond, chelation, another type of chemical interaction, or a combination thereof. Also, for example, a drug may be mixed with carrier, inserted inside a carrier, otherwise interact physically with a carrier so that the carrier encapsulates or otherwise carries the drug, or a combination thereof.

As the term is used herein, “magnetic” refers to something that is a magnet, is capable of becoming a magnet, or is capable of being attracted by a magnet. Accordingly, “magnetized” medications may include materials that are a magnet or materials merely capable of being attracted by a magnet. As the term is used herein, “and/or” refers to two things in the alternative or together, such as “one thing or another thing or both.”

A magnetic field may act on ionized materials to cause movement since they may carry a net positive or negative electrical charge as the result of removing or adding electrons. A magnetic field may also act on magnetic materials to cause movement. Thus, ionic and/or magnetic materials (that is, ionic materials or magnetic materials or both) may be used in the formulation of a drug and/or a carrier to enable use of a magnet to reposition a drug to the selected delivery site in all, or at least most, parts of the brain.

Repositioning may include driving and/or attracting an ionized and/or magnetized medication with the magnetic field. A permanent magnet or another device that generates a magnetic field, such as an electromagnet, may be used. Ionized and/or magnetized materials as carriers could carry a drug, or the drug itself could be ionized and/or magnetized. Ionization and/or magnetization of the drug and/or carrier could occur at manufacture, just prior to administration, or at a time between manufacture and administration. If ionization and/or magnetization occurs early, then attention may be paid to maintaining a suitable level of gauss up until the point of delivery to the target site in the brain.

It should be noted that the described targeted delivery to the brain might not feasible using circulatory delivery methods through infusions, intravenous delivery, or other modalities. Medications in the circulatory system are generally not able to cross the blood-brain barrier (BBB). Thus, the medication may be moved through the nasal cavity utilizing the neuronal pathway in order to bypass the BBB. Without being limited to any particular theory, the neuronal pathway is believed to deliver the drug to the extracellular space in the CSF within the brain cavity, where it is free to travel to desired locations on the brain.

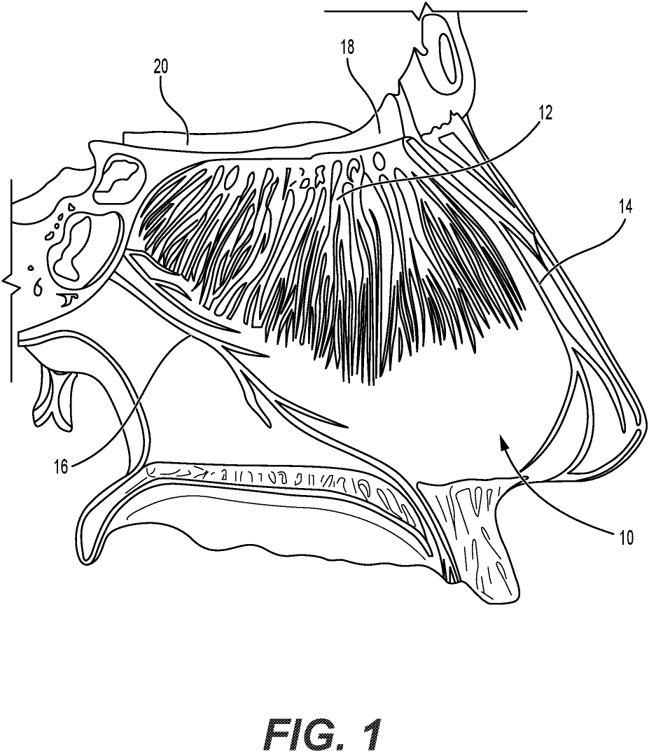

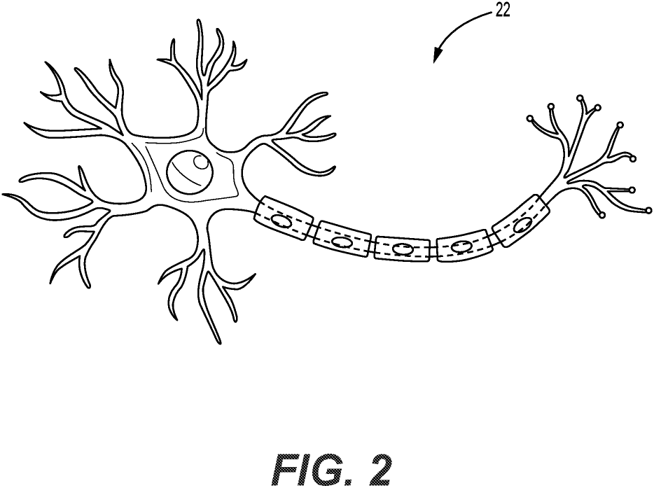

FIG. 1 shows a cross-sectional view of a nasal cavity 10 including selected nerves and other structure relevant to the systems, apparatuses, and methods described herein. Specifically, FIG. 1 shows olfactory nerves 12 extending from an olfactory bulb 20 through a cribriform plate 18 into nasal cavity 10. The brain (not shown) is located adjacent to olfactory bulb 20. Accordingly, it will be appreciated that transport of medication along a neuronal pathway of olfactory nerves 12 may deliver medication from nasal cavity 10 through cribriform 18 into the brain cavity. A neuron 22, shown in FIG. 2, forms a part of olfactory nerves 12 and may play a role in providing the described neuronal pathway. FIG. 1 also shows nasociliary nerves 14 in nasal cavity 10 and nasopalatine nerves 16 in nasal cavity 10. Potentially, nasociliary nerves 14 and/or nasopalatine nerves 16 could participate in a neuronal pathway to the brain cavity.

### Materials and Carriers

Materials that can be ionized and/or magnetic materials can be used to create nanotubes, nanospheres, or other types of nanoscale carriers (herein, “nanocarriers.”). The nanocarriers may be designed to degrade (e.g., dissolve and/or bioabsorb) so that an encapsulated or otherwise carried drug may be released when the nanocarrier degrades.

A nanocarrier is a nanomaterial used as a transport module for another substance. A nanomaterial is a material with any external dimension in the nanoscale or having internal structure or surface structure in the nanoscale. Nanomaterials encompass both nano-objects and nanostructured materials. “Nanoscale” refers to a length range approximately from 1 to less than 1,000 nanometers (nm), such as from 1 to 200 nm, including from 1 to 100 nm.

A microcarrier is a micromaterial used as a transport module for another substance. A micromaterial is a material with any external dimension in the microscale or having internal structure or surface structure in the microscale. Micromaterials encompass both micro-objects and microstructured materials. “Microscale” refers to a length range approximately from 1 to less than 1,000 micrometers (µm), such as from 1 to 200 µm, including from 1 to 100 µm. In selecting a suitable size for a nanocarrier or a microcarrier, consideration may be given to potential size restrictions along an intended pathway from the nose to the brain and then to a selected site on the brain.

A nanocarrier or a microcarrier may be selected from among micelles, polymers, carbon-based materials (e.g., graphene), liposomes, other substances, and combinations thereof. Carbon-based materials may be selected from among carbon nanotubes, carbon nanospheres, other types of carbon nanocarriers, or combinations thereof, including multi-walled carbon nanotubes (MW-CNT).

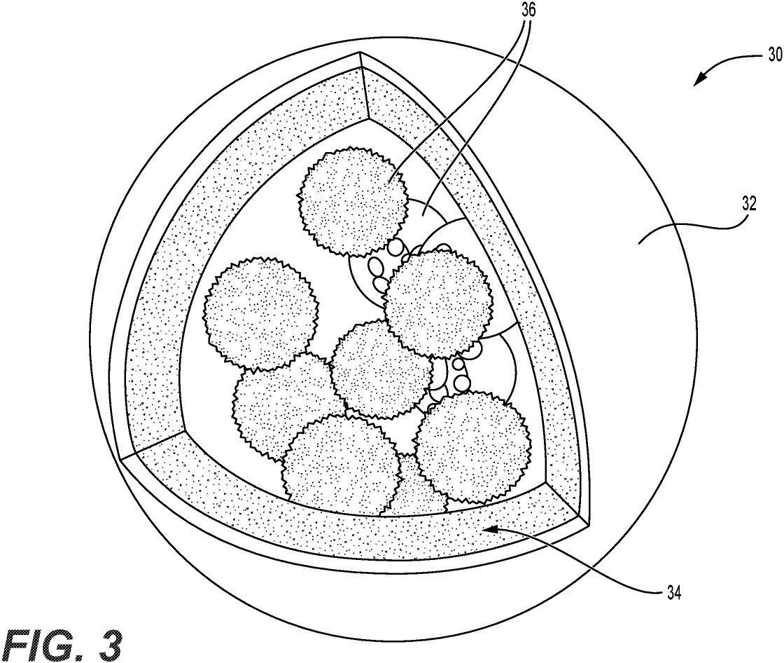

FIG. 3 shows a cut-away, perspective view of one example of a carrier, which may be sized as a nanocarrier or a microcarrier. A carrier 30 in FIG. 3 is layered with an outer layer 32 and an inner layer 34 encapsulating a medication 36. Outer layer 32 may have different electric characteristics compared to inner layer 34 or the same. In that sense, outer layer 32 may ionize or carry a charge to a different degree in comparison to inner layer 34, or to the same degree. Outer layer 32 may have different stability characteristics compared to inner layer 34, such that outer layer 32 may degrade (e.g., dissolve and/or bioabsorb) at a different rate in comparison to inner layer 34. Outer layer 32 may have different magnetic composition in comparison to inner layer 34, such that one might be magnetic to one degree while the other is magnetic to a different degree, or may be the same. Medication 36 may be of a single composition, or carrier 30 may encapsulate medication 36 as a combination of multiple drugs.

As indicated above, FIG. 3 is provided merely as an example. Other examples are possible and may differ from what is described with regard to FIG. 3.

The ability to create superparamagnetic nanoparticles has been established by several industry leaders using a variety of materials for application in many areas of study, including drug delivery. Some of these applications include MRI enhancement and immunoassays, to name a few. Consequently, known superparamagnetic nanoparticles may function as nanocarriers for the medications used in the systems, apparatuses, and methods herein.

Researchers at Lawrence Berkeley National Laboratory created the first permanently magnetic liquids, which opens up new avenues for drug delivery targeting in the brain. In a magnetic liquid, the material retains its magnetic properties in basically any shape. The research showed that magnetic liquid droplets could be divided into multiple smaller droplets, or could be morphed into spheres, cylinders, pancakes, tubes, and even an octopus shape, while still being magnetic. On top of that, the droplets can be tuned so their magnetism can be switched on and off at will.

The use of materials that are magnetized and/or can be ionized, and possibly re-ionized multiple times, may be involved in the targeting and relocating of a drug. Graphene might be used to create nanocarriers that could hold sufficient charge for a long enough periods to reposition in the brain cavity. Others potential carrier materials include chitosan, materials with ionizable hydroxyl group, and materials base on cyanuric acid, among many others. Depending on the drug and disease state, the carrier material can be used to deliver and relocate the drug to a specific place in the brain and potentially other parts of the human body.

It has also been demonstrated that magnetic particles can bind to some regions of antibodies and, thus, can be used to relocate specific target molecules and protein complexes. Magnetic particle applications are not limited by the availability of antibodies. Other ligands such as streptavidin, lectins, enzymes, and other unrelated biological materials can also be used. A major benefit of magnetic nanocarriers and the like is that the aggregations do not need centrifugation with its accompanying stress, allowing a significant increase in the yield of delicately attached protein complexes. Applying a magnet or a magnetic field attracts the molecule bound to a magnetic particle. Accordingly, magnetizing medication could then be done with or without the addition of nano- or microcarriers.

### Delivering to the Brain

Known apparatuses and methods for delivering medication to the brain via the nose may be used. Examples of nose-to-brain medication delivery devices that may be suitable are described in US Pat. No. 9,572,943; No. 8,448,637; No. 8,122,881; No. 9,352,106; No. 7,231,919; No. 7,866,316; No. 7,905,229; No. 8,733,342; No. 10,108,501; and No. 8,001,963, each of which is incorporated herein by reference. The referenced patents also describe medication dispersion devices, such as aerosolizers, included in the medication delivery devices. The suitability of a particular one of the described medication delivery devices may depend on the physical and chemical characteristics of a selected medication. Review of the referenced patents will reveal to ordinarily-skilled persons the suitability of a particular medication delivery device when compared to such physical and chemical characteristics of a medication. Nanotubes were successfully delivered to the brain in a previous study by the technology platform encompassed by the incorporated patents referenced above.

The individual nature of nose-to-brain drug delivery comes from the unique pathway it takes. While it is speculated that the cribriform plate is porous and allows passage of drugs through the bone, it is believed that transport largely occurs through the neuronal pathway at the olfactory region and the trigeminal nerve.

This neuronal pathway would not take the drug into the brain, but rather onto the brain via the perineural, or extracellular, space outside of the cell and directly into the CSF. Transporting the medication in such manner enables it to be relocated because it is not in the brain during transport. Also, the carriers are not in the circulatory system being held and moved by the blood vessels themselves. This also reduces the likelihood that the BBB could be a limiting factor. With time, the brain absorbs the medication into the cells providing the benefit of the medication. However, while the medication is resting on the brain in the CSF, it has the opportunity to be relocated to another part of the brain. Nano or microcarriers encapsulating the drug can enhance the resting time by not degrading immediately, allowing for the drug to be relocated. Time of carrier degradation can be varied through encapsulating material by way of thickness, biodegradable manipulation of the carrier, slowing or accelerating degradation, etc..

### Enhancing the Device

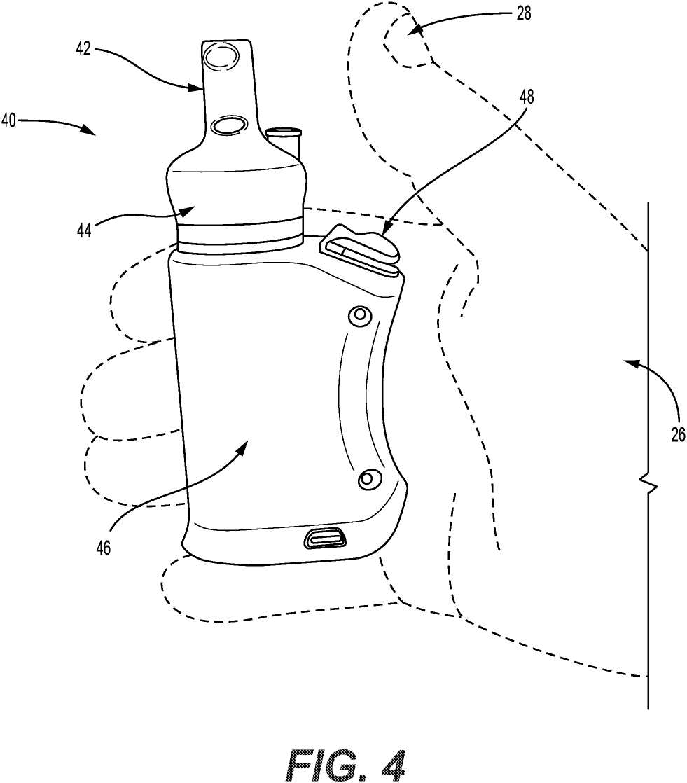

Known medication delivery devices may be adapted to use ionized and/or magnetized medications, such as ionized and/or magnetized drugs and/or carriers. FIG. 4 shows a perspective view of one example of a medication delivery device 40. Device 40 includes a nose piece 42 on top for insertion into the nose. Nose piece 42 is fluidically connected to a chamber 44, wherein medication may be dispersed. Although not shown, chamber 44 forms a part of a medication dispersion device included with device 40. A base unit 46 provides power, mechanical components, and control components sufficient to use ambient air to disperse medication in chamber 44. Base unit 46 also may be hand held by a user. A switch 48 on top of base unit 46 is easily accessible by a thumb 28 of a user’s hand 26. As is described further below, contact of base unit 46 with hand 26 may assist with repositioning the medication.

Adaptations could include making the chamber from a material that can conduct electrical current to the medication. Accordingly, the chamber may add, subtract, or otherwise modulate a charge on the medication with either positive or negative charge depending on the particular medication. As the medication flows through the delivery device from the chamber, subsequently encountered components, such as aerosolization components, if any, may enhance the desired charge characteristics. The base unit of the handheld device may have its own electrical conductivity and control charging of the subject’s body with the same charge as the medication. In this manner, the body maintains the desired charge characteristics by contact with the medication so the medication can be repositioned by the magnetic field. Body charging may be turned off depending on characteristics of the medication.

As indicated above, FIG. 4 is provided merely as an example. Other examples are possible and may differ from what is described with regard to FIG. 4.

### External Headgear

Once the ionized and/or magnetized medication has been delivered to the brain, the dose can be moved to a new location by way of external magnetic field generators worn on the head of the patient. In one implementation, a central control module may contain a programming interface and power (AC and/or battery). A plurality of arms, upon which is mounted one or more magnetic field generators, may extend down from the central module. The magnetic field generator may include plurality of magnets and/or electromagnets that can be adjusted along the length of the arms.

The central control module may include a programmable interface that communicates with a computer through an interface, possibly a USB port or another interface, including a wireless interface. Programming may activate a plurality of electromagnets in series or parallel time sequencing according to instructions given by the program. In parallel time sequencing, both activate at the same time. In series time sequencing, one activates after the other.

In the example of a brain tumor, results of magnetic resonance imaging (MRI) identifying the tumor location may be overlayed on the subject’s head with a grid. The physician may identify where a dose of medication should be repositioned by selecting the grid squares that correspond to the tumor location. Grid square selections may inform a technician where to place the magnetic field generators and which ones to activate at what time throughout the medication delivery and repositioning.

Arms of the headgear may be adjustable in three dimensions and spring loaded to be retracted until extended to be placed on the subject’s head. Once placed in the correct position, the drug delivery device may be activated in parallel or in series time sequencing with the programming of the headgear.

The number of magnets is limited only by the space available on the head. It is anticipated that pediatric-sized headgear may be useful to permit precision placement as well as dose timing and onset of action. The magnetic field generators may be capable of static operation and/or pulsing for adjusting speed of repositioning. A driving magnetic field generator pushing the medication toward the target site could be pulsed, while a drawing magnetic field generator may be constantly on until completion.

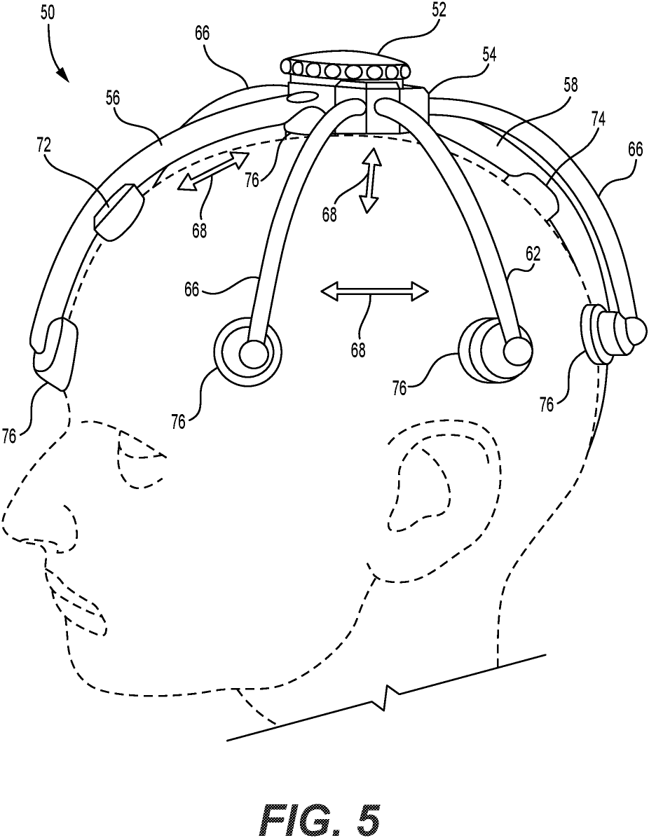

FIG. 5 shows a perspective view of an example headgear 50 described herein. Example headgear 50 includes a base 54 configured for locating over a top of the brain when in use. A front arm 56, a rear arm 58, a left arm 62, and a right arm 64 (not shown in FIG. 5) each have an arcuate longitudinal shape. Each of front arm 56, rear arm 58, left arm 62, and right arm 64 are connected to base 54. Also, each extends over a side of the brain when in use (positioned on the head).

FIG. 5 also shows three intermediate arms 66 and head 50 includes a fourth intermediate arm 66 not apparent in FIG. 5. Each of intermediate arms 66 are placed between two of the other arms. That is, one intermediate arm 66 is between front arm 56 and left arm 62. Another intermediate arm 66 is between left arm 62 and rear arm 58. A further intermediate arm 66 (not shown) is between rear arm 58 and right arm 64. A further intermediate 66 is between right arm 64 and front arm 56. Intermediate arms 66 are shaped similarly to arms 56, 58, 62, and 64.

While each arm is connected to base 54, an end of each of arms 56, 58, 62, 64, and 66 is located distally from base 54 over a side of the brain when in use. A pad 76 is placed at the respective ends of each of the arms in a position selected to contact the head such that each pad 76 acting together holds headgear 50 in place. A pad 76 is also provided between base 54 and the head.

Headgear 50 includes a front magnet 72 and a rear magnet 74 in the example shown. Front magnet 72 is releasably secured on front arm 56 and rear magnet 74 is releasably secured on rear arm 58. With the magnets releasably secured, they may be relocated to different positions on front arm 56 or rear arm 58 or other arms, such as left arm 62, right arm 64, and intermediate arms 66.

The magnets may generate a magnetic field of sufficient strength and direction to reposition a medication to a selected drug delivery site on the brain following initial delivery of the medication from a medication delivery device to the brain cavity via the nose while bypassing the blood-brain barrier. Arrows 68 show that front magnet 72 and rear magnet 74 may be positioned within three dimensions by moving the magnets or moving the arms. Each of arms 56, 58, 62, 64, and 66 may be rotated such that any position on the surface area of the head may be reached by one of the arms and a corresponding magnet placed at such position.

As indicated above, FIG. 5 is provided merely as an example. Other examples are possible and may differ from what is described with regard to FIG. 5.

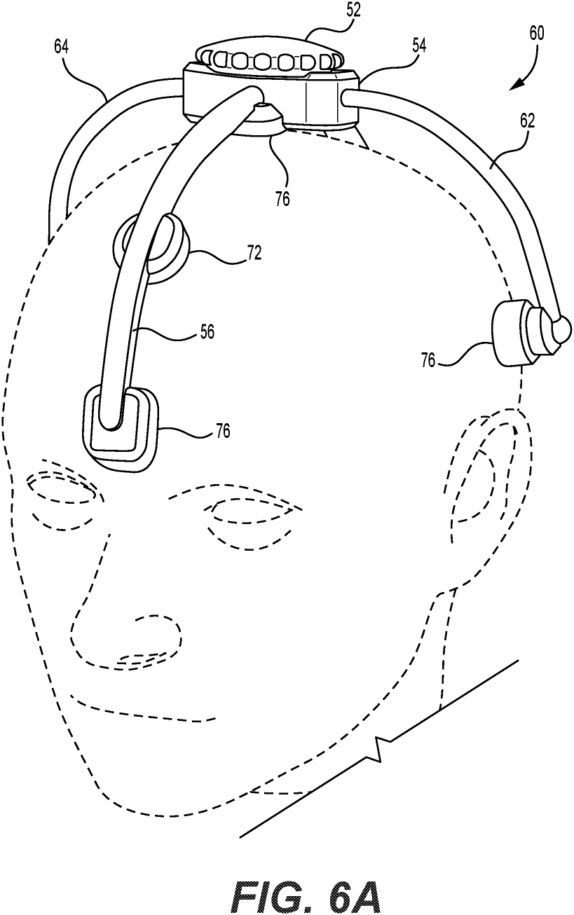

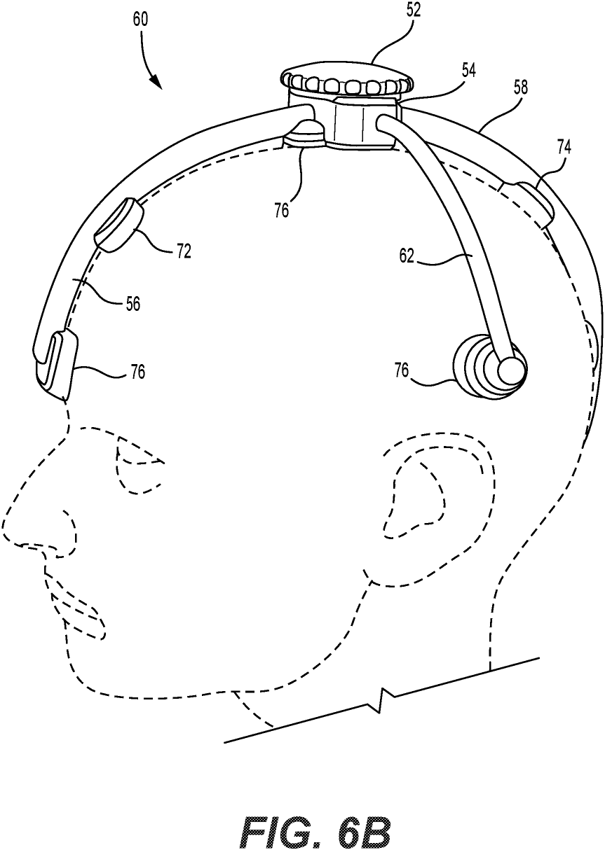

FIGS. 6A-6B show a perspective view of another example headgear 60 described herein. Headgear 60 is the same as headgear 50 except that four intermediate arms 66 are removed. FIG. 6A also shows right arm 64, which does not appear in FIG. 5. It will be appreciated that the full coverage afforded by the eight arms in FIG. 5 might not be required in all applications. Headgear 60 in FIGS. 6A-B may provide adequate coverage even though it does not provide full coverage.

Headgear 50 and headgear 60 both include a control module 52. In FIGS. 5 and 6A-B, front magnet 72 and rear magnet 74 are shown to generate a magnetic field. Alternatively or additionally, one or more electromagnets may be used, the operation of which is controlled by control module 52. Accordingly, while front magnet 72 and rear magnet 74 provide a constant magnetic field, the magnetic field of an electromagnet may be selectively switched off.

As indicated above, FIGS. 6A-B are provided merely as an example. Other examples are possible and may differ from what is described with regard to FIGS. 6A-B.

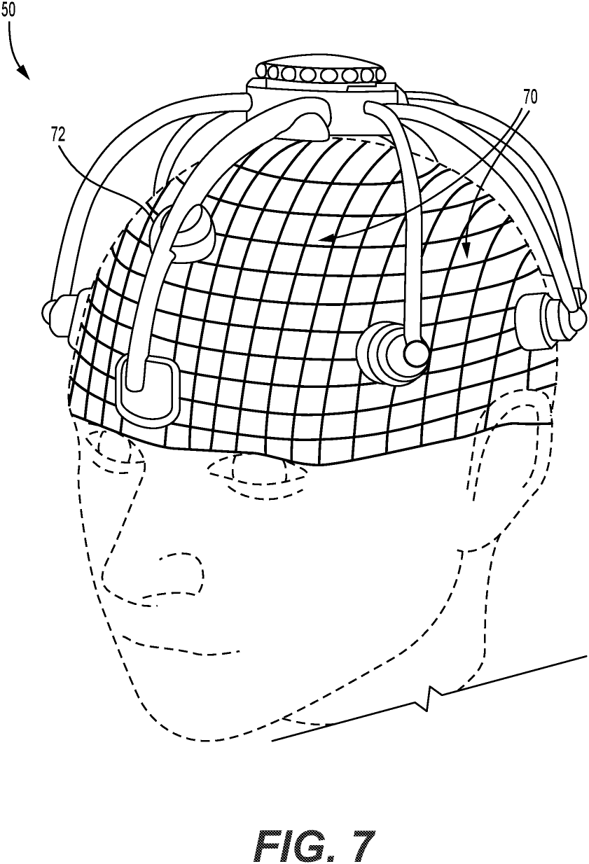

FIG. 7 shows a perspective view of head gear 50 in an example implementation in combination with a grid 70. Headgear 50 in FIG. 7 includes all of the features of headgear 50 in FIG. 5. The example implementation of FIG. 7 additionally shows grid 70 overlaid on the head to permit accurate locating of front magnet 72 or some other magnetic field generator that may be used with headgear 50. Relying on imaging of the brain, a physician may select a location for repositioning medication on the brain. The selected location will determine the positioning of magnetic field generators. Grid 70 simplifies and increases the accuracy of following the physician’s instructions for magnet placement.

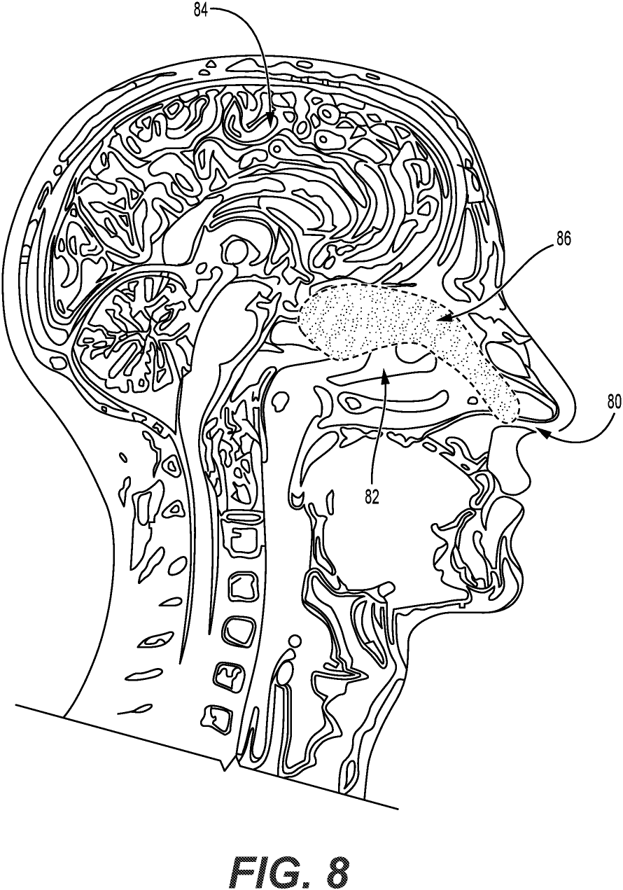

FIG. 8 show a hypothetical image of a scan taken on a subject who received a magnetized medication 86 through a nose 80 into a nasal cavity 82. FIG. 8 also shows a portion of medication 86 delivered into a portion of a brain 84 adjacent nasal cavity 82.

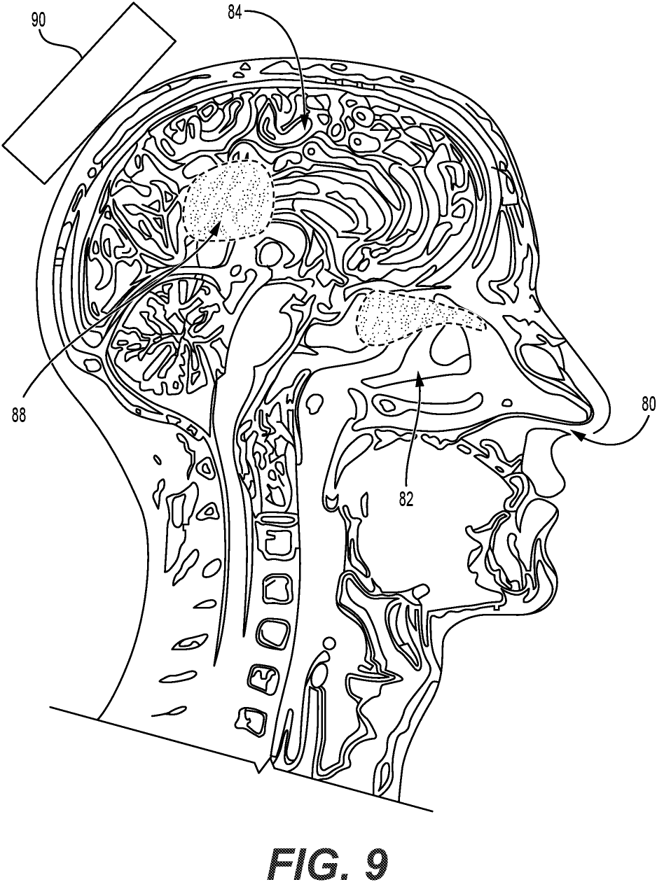

FIG. 9 shows medication 88 repositioned from nasal cavity 82 and the portion of brain 84 adjacent to nasal cavity 82 to a new position in brain 84 as directed by attracting magnet 90. FIG. 9 thus provides an example of repositioning medication 86 from the location FIG. 8 to the location shown in FIG. 9 with a magnetic field generator. Magnetized medication 86 allows a single magnetic field generator, such as attracting magnet 90, to reposition a portion of medication 86 to the location shown by medication 88.

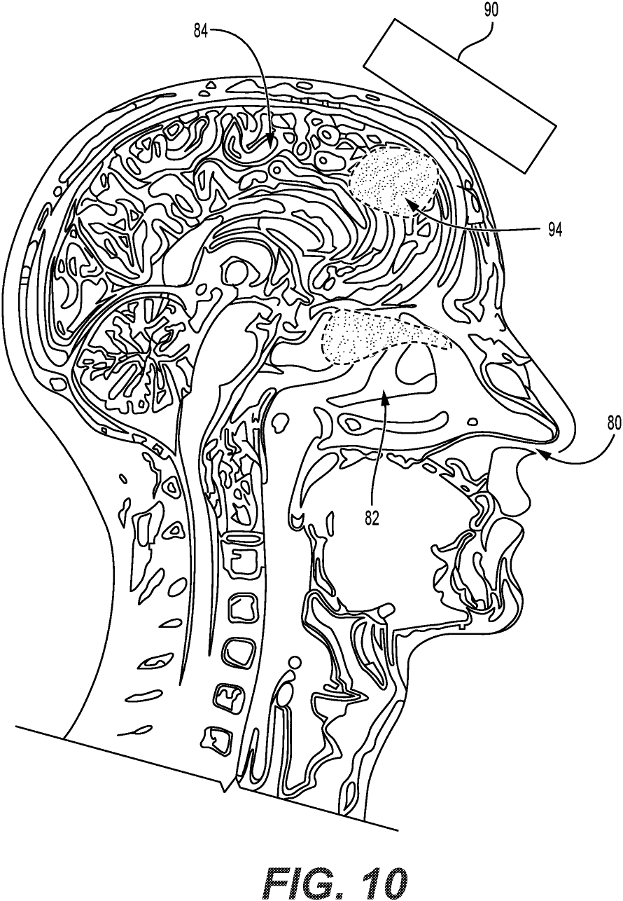

FIG. 10 similarly shows attracting magnet 90 in a different location that repositioned a portion of medication 86 to the location shown for medication 94. In the hypothetical examples of FIGS. 8-10, merely changing the location of attracting magnet 90 shifts its magnet field such that magnetized medication 86 is “attracted” toward attracting magnet 90.

As indicated above, FIGS. 8-10 are provided merely as an example. Other examples are possible and may differ from what is described with regard to FIGS. 8-10.

Settings may vary considerably for greatest efficiency in reaching a particular target site or multiple sites and repositioning a particular medication. For example, drugs will naturally migrate to the brain using the neuronal pathway in the nasal cavity in about 30 minutes. This could be accelerated by using a magnetic field generator positioned above the nose on the head. The generator may draw the medication into the extracellular space in a shorter period of time. Once migration from the nasal cavity to the brain has occurred, the initial drawing magnet is turned off and a driver magnet of the same polarity is switched on in order to drive the carrier to the target site. In parallel operation, another magnet of the opposite polarity is also turned on to draw the carrier to the target site. Depending on the distance of the initial migration of carriers from the target site, multiple magnets may be used to move the dose to remote regions of the brain as determined by the initial migration location.

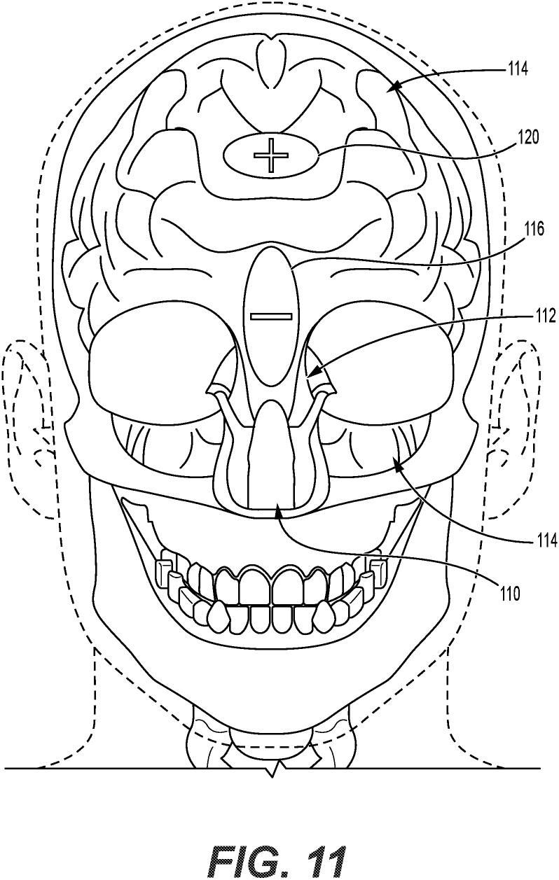

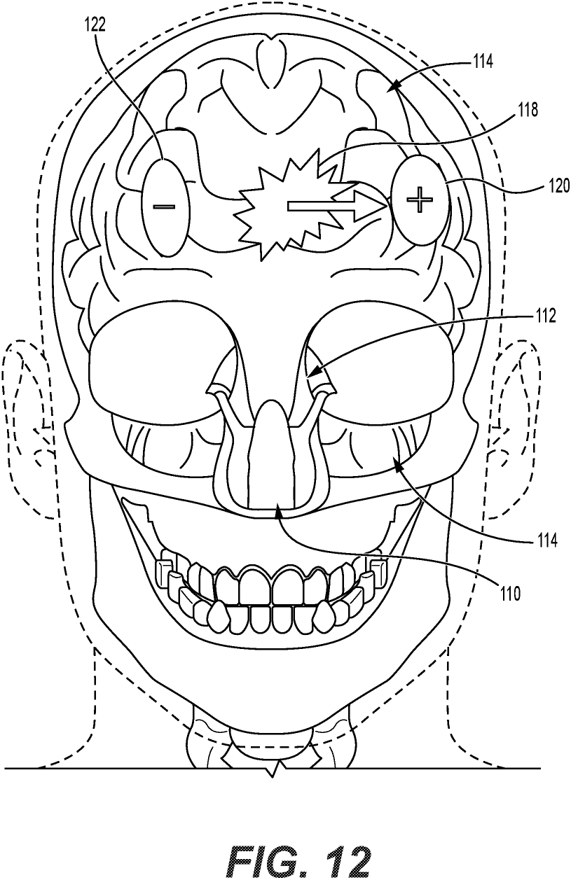

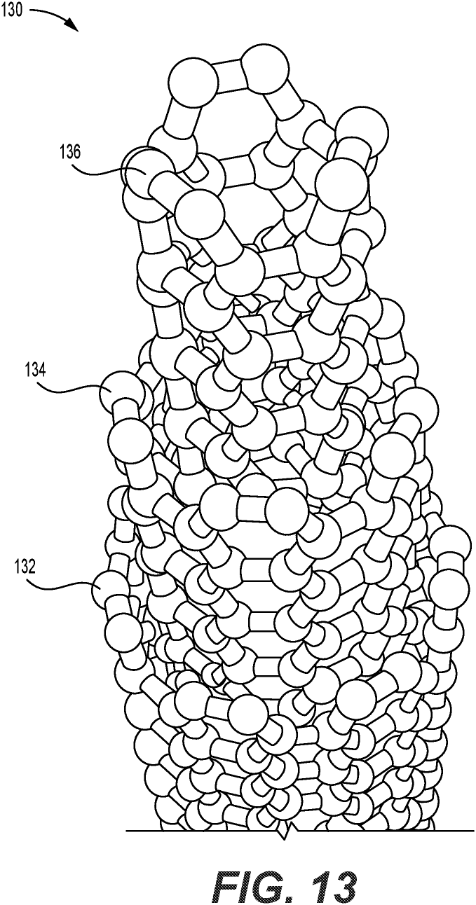

While FIGS. 8-10 provide a simple example of repositioning magnetized medication with one magnet, FIGS. 11 and 12 provide a graphically-depicted example of using two magnets. In the example of FIG. 11, a medication 116 is shown in a nasal cavity 112 following introduction via a nose 110. FIG. 11 depicts a polarity of the magnetization of medication 16 with a “minus” sign. To facilitate delivery of medication 116 onto brain 114, an attracting magnet 120 is provided at the position shown in FIG. 11. The magnetic polarity of attracting magnet 120 is depicted in FIG. 11 with a “plus” sign, opposite to the polarity of medication 116.

With the passage of time, FIG. 12 shows medication 116 repositioned to the location shown for a medication 118 in FIG. 12. FIG. 12 also shows the relocation of attracting magnet 120 and the addition of a driving magnet 122. The magnetic polarity of driving magnet 122 is depicted in FIG. 12 with a “minus” sign, the same as the polarity of medication 116 and 118. Driving magnet 122 with the same polarity drives medication 118 away from the location of driving magnet 122 in the direction shown by the arrow superimposed over medication 118. In contrast, attracting magnet 120 attracts magnetized medication 118 of opposite polarity in the same direction as shown by the arrow superimposed over magnetized medication 118.

Accordingly, by shifting the location and number of magnets, FIGS. 11 and 12 provide one example of delivering medication onto the brain at one location followed by repositioning to another location. Although the examples FIGS. 11 and 12 rely upon magnetized medication, a magnetic field also acts upon ionized medication. While the relative positioning of magnets and the most suitable strength and direction of a magnetic field may differ for ionized medication, the principles explained in association with FIGS. 8-12 are similar.

As indicated above, FIGS. 8-12 are provided merely as an example. Other examples are possible and may differ from what is described with regard to FIGS. 8-12.

It has been established that, using a neodymium magnet, magnetized particles have been measured to migrate 1 centimeter every 2 minutes. An electromagnet can be adjusted as to power and location and could accelerate this process. The headgear can be powered by batteries or by plugging it into standard AC power.

When the medication is delivered with a certain magnetization polarity, it may be drawn by a magnet of the opposite polarity worn by the patient so that it attracts the medication to the site in the brain or in the body where the medication is needed. Once there, the system can detect the change in the magnetic field when the opposing poles become adjacent and the electromagnet may be turned off, halting the migration of the medication. A series of magnets of the same polarity can be used to drive the medication toward the magnet of opposite polarity at the target site.

For nose-to-brain delivery, it may be beneficial to ionize and re-ionize the medication so it can be moved multiple times. In addition, materials may be used for carriers that are inert until placed in a magnetic field. This property could allow the use of electromagnets to turn the carriers “on” and “off” as time passes.

As mentioned previously, carriers may be created of varying thicknesses and/or use materials for which the degradation rates are variable, but established. As an example, carriers may be selected to allow some of the medications to be released from some of the carriers, while others remain encapsulated. Once the appropriate period of time has passed, the electromagnets can be restarted, and the remaining carriers can be moved to a different location. This approach allows a non-invasive way of treating multiple sites with a single dose.

Implementation may use carriers of varying thicknesses or several layers to the carrier that release different medication as each successive layer degrades. This can be controlled release or it can be a partial release and then a separate migration once the first layer has delivered. FIG. 11 shows a perspective, magnified view of a multiple-walled nanotube 130. An intermediate wall 134 is between an inner wall 136 and an outer wall 132. The nanotube walls provide one example of a carrier having several layers, as described above.

In some cases, the gear worn by the patient may be capable of re-ionizing the medication through the body and then drawn further by an opposite polarity to the next medication target site.

### Materials and Nanocarriers

Medications have been produced and delivered to the body through the encapsulation of medication in a microsphere, nanosphere, microtube, nanotube, or other medication-encasing substance or form. The outer layer of the carrier may be capable of holding a charge, and also may be degraded by the body over time, releasing the drug as the outer layer degrades. These materials may also be capable of being re-ionized to ensure the delivery of the drug to the target site.

Multiple layers can be produced of the same or different materials that are magnetized and/or ionized separately in production as well as inside the human body or head. When the outer layer has completely degraded, the external body gear may be placed at the site and the outer layer may be re-ionized if needed and then driven from behind/above/below as needed while simultaneously being drawn by the opposite polarity to the ultimate drug target site. The ability to move the carriers can be done as many times as advisable prior to the last layer of the casing degrading and releasing the medication.

Targeted magnetic particles can be used in other parts of the body as long as the circulatory system or the gastrointestinal tract are not used to deliver the drug. Treatment of pancreatic cancer may be one example, directly injecting the encapsulated and/or magnetized drug onto the organ and use external magnetic fields to move the carriers. The carriers having freedom of movement and not being bound by the anatomy increases the effectiveness. As a second example, chronic obstructive pulmonary disease could be better served by using magnetic nanocarriers and drawing the drugs deeper into the lungs, thus maximizing the amount of drug that is delivered to the areas that are most beneficial to the patient, but difficult or impossible to reach by current methods.

### Systems, Apparatuses, and Methods

The discoveries described herein identify a number of solutions that may be implemented in systems, apparatuses, and methods also described herein. Multiple solutions may be combined for implementation, enabling still further systems, apparatuses, and methods. The inventors expressly contemplate that the various options described herein for individual systems, apparatuses, and methods are not intended to be so limited except where incompatible with other systems, apparatuses, and methods. The features and benefits of individual systems and apparatuses herein may also be used in combination with methods and other systems and apparatuses described herein even though not specifically indicated elsewhere. Similarly, the features and benefits of individual methods herein may also be used in combination with systems and apparatuses and other methods described herein even though not specifically indicated elsewhere.

System A includes a nose-to-brain medication delivery device including a medication dispersion device and a magnetic field generator. The generator, when in use, generates a magnetic field of sufficient strength and direction to reposition an ionized and/or magnetized medication to a selected drug delivery site on the brain following initial delivery of the medication from the medication delivery device to the brain cavity via the nose while bypassing the blood-brain barrier.

Additional features may be implemented in System A. By way of example, the medication dispersion device may include an aerosolizer. The medication dispersion device may use ambient air to disperse the medication.

System A may further include a medication that is ionized, magnetized, or both, or configured to be ionized, magnetized, or both by the medication delivery device during dispersion, after dispersion, or both by the medication dispersion device. The medication may include a carrier and the carrier may be ionized, magnetized, or both, or configured to be ionized, magnetized, or both by the medication delivery device during dispersion, after dispersion, or both by the medication dispersion device. The carrier may include a nanocarrier or a microcarrier. The medication may include a composition that may be re-ionized while on the brain.

System A may further comprise a headgear apparatus, which includes a base configured for locating over a top of the brain when in use and a front arm, a rear arm, a left arm, and a right arm each having an arcuate longitudinal shape. Each may be connected to the base and each may extend over a side of the brain when in use. An end of each of the front, rear, left, and right arms may be located distally from the base over a side of the brain when in use. The magnetic field generator may be releasably secured on at least one of the front, rear, left, and right arms. The magnetic field generator may include one or more electromagnets, one or more permanent magnets, or both.

The described additional features of System A may also be implemented in other system, apparatuses, and methods herein.

Apparatus B is a headgear including a base configured for locating over a top of the brain when in use and a front arm, a rear arm, a left arm, and a right arm each having an arcuate longitudinal shape. Each is connected to the base and each extends over a side of the brain when in use. An end of each of the front, rear, left, and right arms located distally from the base over a side of the brain when in use. A magnetic field generator is releasably secured on at least one of the front, rear, left, and right arms. The magnetic field generator is configured to generate, when in use, a magnetic field of sufficient strength and direction to reposition an ionized and/or magnetized medication to a selected drug delivery site on the brain following initial delivery of the medication from a medication delivery device to the brain cavity via the nose while bypassing the blood-brain barrier.

Additional features may be implemented in Apparatus B. By way of example, the base may include a control module and the magnetic field generator may include one or more electromagnets, the operation of which is controlled by the control module. The magnetic field generator may include one or more permanent magnets.

The described additional features of Apparatus B may also be implemented in other devices and methods herein.

Method C includes a medication dispersion device dispersing a medication in a gas and ionizing and/or magnetizing the medication before, during, or after the dispersing. A nose-to-brain medication delivery device delivers the dispersed medication that is ionized and/or magnetized to the brain cavity via the nose while bypassing the blood-brain barrier. Method C includes applying a magnetic field to the brain and thereby repositioning the ionized and/or magnetized medication to a selected drug delivery site on the brain following the delivery of the medication from the medication delivery device to the brain cavity.

Additional features may be implemented in Method C. By way of example, the dispersing of the medication may include aerosolizing the medication. The gas may be ambient air.

The medication may include a carrier and the ionizing and/or magnetizing of the medication may include ionizing and/or magnetizing the carrier. The carrier may include a nanocarrier or a microcarrier. The method may further include re-ionizing the medication while on the brain.

Method C may further include placing a headgear apparatus over the brain, wherein the magnetic field is applied to the brain using the headgear apparatus. The headgear may include a base configured for locating over a top of the brain and a front arm, a rear arm, a left arm, and a right arm each having an arcuate longitudinal shape. Each may be connected to the base and each may extend over a side of the brain. An end of each of the front, rear, left, and right arms may be located distally from the base over a side of the brain. A magnetic field generator may be releasably secured on at least one of the front, rear, left, and right arms. The application of the magnetic field may include using one or more electromagnets, one or more permanent magnets, or both.

The described additional features of Method C may also be implemented in other systems, apparatuses, and methods herein.

Although minima and maxima are listed for the above described ranges and other ranges designated herein, it should be understood that more narrow included ranges may also be desirable and may be distinguishable from prior art. Also, processing principles discussed herein may provide an additional basis for the lesser included ranges.

In compliance with the statute, the embodiments have been described in language more or less specific as to structural and methodical features. It is to be understood, however, that the embodiments are not limited to the specific features shown and described. The embodiments are, therefore, claimed in any of their forms or modifications within the proper scope of the appended claims appropriately interpreted in accordance with the doctrine of equivalents.

TABLE OF REFERENCE NUMERALS FOR FIGURES

| | |
|---|---|
| 10 | nasal cavity |
| 12 | olfactory nerves |
| 14 | nasociliary nerves |
| 16 | nasopalatine nerves |
| 18 | cribriform plate |
| 20 | olfactory bulb |
| 22 | neuron |
| 26 | hand |
| 28 | thumb |
| 30 | carrier |
| 32 | outer layer |
| 34 | inner layer |
| 36 | medication |
| 40 | device |
| 42 | nosepiece |
| 44 | chamber |
| 46 | base unit |
| 48 | switch |
| 50 | headgear |
| 52 | control module |
| 54 | base |
| 56 | front arm |
| 58 | rear arm |
| 60 | headgear |
| 62 | left arm |
| 64 | right arm |
| 66 | intermediate arm |
| 68 | arrow |
| 70 | grid |
| 72 | front magnet |
| 74 | rear magnet |
| 76 | pad |
| 80 | nose |
| 82 | nasal cavity |
| 84 | brain |
| 86 | medication |
| 88 | medication |
| 90 | attracting magnet |
| 94 | medication |
| 110 | nose |
| 112 | nasal cavity |
| 114 | brain |
| 116 | medication |
| 118 | medication |
| 120 | attracting magnet |
| 122 | driving magnet |
| 130 | nanotube |
| 132 | outer wall |
| 134 | intermediate wall |
| 136 | inner wall |

What is claimed is:

1. A system comprising:
a nose-to-brain medication delivery device including a medication dispersion device; and
a magnetic field generator configured to generate, when in use, a magnetic field of sufficient strength and direction to reposition an ionized and/or magnetized medication to a selected drug delivery site on the brain following initial delivery of the medication from the medication delivery device to the brain cavity via the nose while bypassing the blood-brain barrier.

2. The system of claim 1, wherein the medication dispersion device comprises an aerosolizer.

3. The system of claim 1, wherein the medication dispersion device uses ambient air to disperse the medication.

4. The system of claim 1, further comprising a medication that is ionized, magnetized, or both, or configured to be ionized, magnetized, or both by the medication delivery device during dispersion, after dispersion, or both by the medication dispersion device.

5. The system of claim 4, wherein the medication includes a carrier and the carrier is ionized, magnetized, or both, or configured to be ionized, magnetized, or both by the medication delivery device during dispersion, after dispersion, or both by the medication dispersion device.

6. The system of claim 5, wherein the carrier comprises a nanocarrier or a microcarrier.

7. The system of claim 4, wherein the medication includes a composition that may be re-ionized while on the brain.

8. The system of claim 1, further comprising a headgear apparatus, which comprises:
a base configured for locating over a top of the brain when in use;
a front arm, a rear arm, a left arm, and a right arm each having an arcuate longitudinal shape, each being connected to the base, and each extending over a side of the brain when in use;
an end of each of the front, rear, left, and right arms located distally from the base over a side of the brain when in use; and
the magnetic field generator is releasably secured on at least one of the front, rear, left, and right arms.

9. The system of claim 1, wherein the magnetic field generator comprises one or more electromagnets, one or more permanent magnets, or both.

10. A headgear apparatus comprising:
a base configured for locating over a top of the brain when in use;
a front arm, a rear arm, a left arm, and a right arm each having an arcuate longitudinal shape, each being connected to the base, and each extending over a side of the brain when in use;
an end of each of the front, rear, left, and right arms located distally from the base over a side of the brain when in use; and
a magnetic field generator releasably secured on at least one of the front, rear, left, and right arms and configured to generate, when in use, a magnetic field of sufficient strength and direction to reposition an ionized and/or magnetized medication to a selected drug delivery site on the brain following initial delivery of the medication from a medication delivery device to the brain cavity via the nose while bypassing the blood-brain barrier.

11. The headgear apparatus of claim 10, wherein the base comprises a control module and the magnetic field generator comprises one or more electromagnets, the operation of which is controlled by the control module.

12. The headgear apparatus of claim 10, wherein the magnetic field generator comprises one or more permanent magnets.

13. A method comprising:
a medication dispersion device dispersing a medication in a gas;
ionizing and/or magnetizing the medication before, during, or after the dispersing;
a nose-to-brain medication delivery device delivering the dispersed medication that is ionized and/or magnetized to the brain cavity via the nose while bypassing the blood-brain barrier; and
applying a magnetic field to the brain and thereby repositioning the ionized and/or magnetized medication to a selected drug delivery site on the brain following the delivery of the medication from the medication delivery device to the brain cavity.

14. The method of claim 13, wherein the dispersing of the medication comprises aerosolizing the medication.

15. The method of claim 13, wherein the gas is ambient air.

16. The method of claim 13, wherein the medication includes a carrier and the ionizing and/or magnetizing of the medication comprises ionizing and/or magnetizing the carrier.

17. The method of claim 13, wherein the carrier comprises a nanocarrier or a microcarrier.

18. The method of claim 13, wherein the method further comprises re-ionizing the medication while on the brain.

19. The method of claim 13, further comprising placing a headgear apparatus over the brain, wherein the magnetic field is applied to the brain using the headgear apparatus, which comprises:
a base configured for locating over a top of the brain;
a front arm, a rear arm, a left arm, and a right arm each having an arcuate longitudinal shape, each being connected to the base, and each extending over a side of the brain;
an end of each of the front, rear, left, and right arms located distally from the base over a side of the brain; and
a magnetic field generator releasably secured on at least one of the front, rear, left, and right arms.

20. The method of claim 13, wherein the application of the magnetic field comprises using one or more electromagnets, one or more permanent magnets, or both.
